(19) United States
(12) Patent Application Publication
(10) Pub. No.: US 2019/0124983 A1
(43) Pub. Date: May 2, 2019

(54) MICRO-VAPORIZER WITH LEAK PROTECTION

(71) Applicant: Avail Vapor, LLC, Richmond, VA (US)

(72) Inventors: Russell Paul Rogers, Richmond, VA (US); Daniel Donovan Harlin Phillips, Richmond, VA (US)

(21) Appl. No.: 16/114,758

(22) Filed: Aug. 28, 2018

Related U.S. Application Data
(60) Provisional application No. 62/580,512, filed on Nov. 2, 2017.

(51) Int. Cl. A24F 47/00

(52) U.S. Cl. CPC A24F 47/008; A61M 15/06

(57) ABSTRACT

A micro-vaporizer has a main body in which are disposed a vaporization chamber and a vaporizable liquid reservoir. A heating element disposed within the vaporization chamber is configured to vaporize liquid drawn from the liquid reservoir. An air flow passage from an inlet portal to the vaporization chamber provides a first fluid communication path between the vaporization chamber and an ambient environment external to the main body. A vaporization products flow passage from the vaporization chamber to an exit port provides a second fluid communication path between the vaporization chamber and the ambient environment. An air-permeable liquid barrier disposed within the air flow passage is configured to inhibit passage of the vaporizable liquid through the air flow passage.

This application claims priority to U.S. Provisional No. 62/580,512, filed Nov. 2, 2017, the complete disclosure of which is incorporated herein by reference.

### BACKGROUND OF THE INVENTION

The present invention relates generally to micro-vaporizers and, more particularly, to micro-vaporizers having a mechanism for reducing or eliminating leakage of vaporizable liquid.

Micro-vaporizers are devices in which a vaporizable liquid is drawn from a storage reservoir into a chamber where it is heated to vaporization temperature by a heating element. The vaporized liquid is then drawn or forced from the chamber. In products such as electronic cigarettes (also known as e-cigarettes or personal vaporizers), the vaporized liquid is drawn from the chamber through a mouthpiece and inhaled by the user. In other products the vaporized liquid is dispersed into the atmosphere.

The usual purpose of a device that uses a micro-vaporizer is to dispense one or more active substances using the vaporized liquid. In atmospheric dispensers, these substances may include materials such as deodorizing agents, fragrance, and insect repellant. In the case of personal vaporizers, the active substances typically include a flavorant (i.e., a flavoring agent or material) and nicotine. The flavorant and nicotine levels may be selected so as to mimic the experience of smoking a cigarette.

A recurring problem with many personal vaporizers is the tendency for the vaporizable liquid to migrate from the reservoir when the heating element is not activated. This can result in the liquid flowing into and through the air passages of the device, which can result in liquid leaking out of the device through its air intake ports and/or its mouthpiece port.

### SUMMARY OF THE INVENTION

An illustrative aspect of the invention provides a micro-vaporizer comprising a main body having a main body interior. The micro-vaporizer further comprises a vaporization chamber and a vaporizable liquid reservoir, both disposed within the main body interior. The vaporizable liquid reservoir is configured for selectively retaining a vaporizable liquid. The micro-vaporizer further comprises a heating element disposed within the vaporization chamber and configured to selectively heat and vaporize vaporizable liquid drawn from the liquid reservoir. The micro-vaporizer still further comprises one or more air inlet openings in the main body collectively defining an air intake portal and an air flow passage from the air intake portal to the vaporization chamber. The air flow passage provides a first fluid communication path between the vaporization chamber and an ambient environment external to the main body. The vaporizer also has a vaporization products flow passage from the vaporization chamber to an exit port, the vaporization products flow passage providing a second fluid communication path between the vaporization chamber and the ambient environment external to the main body. The vaporizer also comprises a first air-permeable liquid barrier disposed within the air flow passage. The first air-permeable liquid barrier is configured to inhibit passage of the vaporizable liquid through the air flow passage.

### BRIEF DESCRIPTION OF THE DRAWINGS

The invention can be more fully understood by reading the following detailed description together with the accompanying drawing, in which like reference indicators are used to designate like elements, and in which:

FIG. 1 is an exploded perspective view of a personal vaporizer according to an embodiment of the invention;

FIG. 2 is a partially sectioned view of a personal vaporizer according to an embodiment of the invention;

FIG. 3 is a full sectioned view of the personal vaporizer of FIG. 2; and

FIG. 4 is a full sectioned view of a personal vaporizer according to an embodiment of the invention.

### DETAILED DESCRIPTION OF THE INVENTION

The present invention provides micro-vaporizers in which liquid leakage is reduced or eliminated through the use of one or more screening mechanisms that inhibit the flow of liquid through the passages of the micro-vaporizer while allowing the flow of air and/or combinations of air and vapor. These screening mechanisms may be or include porous media placed in different locations within the micro-vaporizer. Such media may be tailored to retain or repel different types of liquid, depending on their particular purpose or application.

In each of various embodiments of the invention, a micro-vaporizer comprises a case or main body in which is disposed a vaporizable liquid source from which vaporizable liquid, typically comprising one or more active materials, is drawn to or is otherwise presented to a heat source that causes the liquid to be vaporized. The resulting vapor is mixed with air in a vaporization chamber, then passed to an exit chamber where it exits the device. In typical personal vaporizers, the exit chamber is defined by a mouthpiece (sometimes referred to as a “tip” or “drip tip”) and the combined air/vapor mixture is drawn through and out of the device by inhalation by a user. The case may be a single monolithic structure or may be made up of multiple sub-structures.

As used herein, the term “active material” refers to any material that controllably alters or adds to the vaporization products of the device. Depending on the application, active materials can include, without limitation, plant material, minerals, deodorizing agents, fragrances, insect repellants, medications, and disinfectants and any material or structure containing or incorporating any of the foregoing.

In the specific instance of personal vaporizers, active materials may include flavorant substances that augment the flavorant of the vaporizable liquid. These may include, without limitation, marijuana, hemp, cannabidiol (cbd), citronella, geraniol, mint, thyme, tobacco, salvia dorrii, salvia, passiflora incarnata, arctostaphylos uva-ursi, lobelia inflata, lemon grass, cedar wood, clove, cinnamon, coumarin, helio, vanilla, menthol, eucalyptus, peppermint, rosemary, lavender, licorice, and cocoa and any material or structure containing or incorporating any of the foregoing.

The invention will be described in more detail using examples and embodiments geared primarily to personal vaporizers. It will be understood, however, that the methods of the invention are not limited to such applications and can be applied to any micro-vaporizer device.

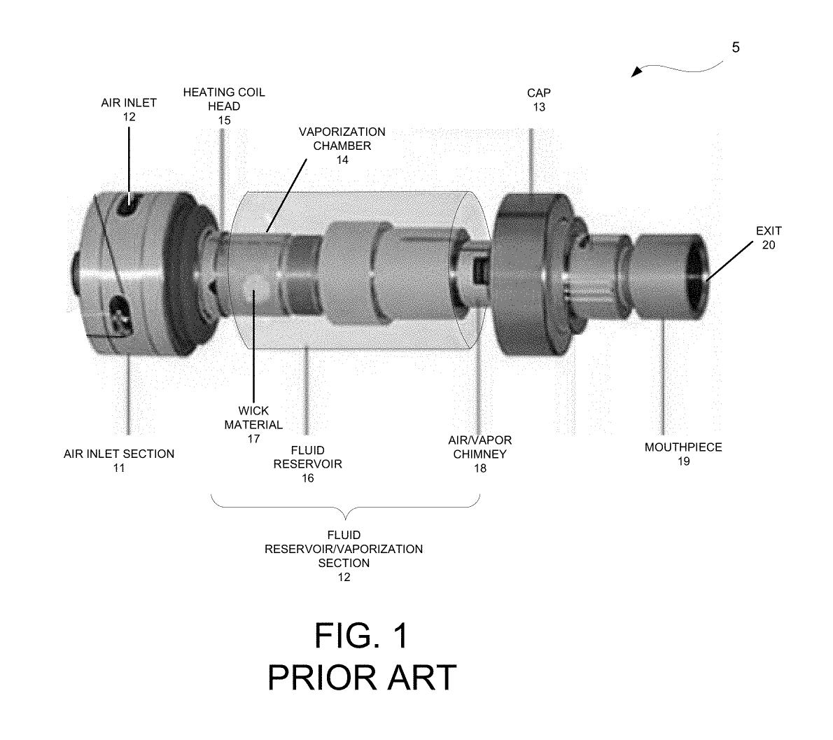

FIG. 1 is a partially exploded view of a typical personal vaporizer 5 having a main body comprising an air inlet section 11, a liquid reservoir/vaporization chamber section 12, and a cap 13. A mouthpiece section 19 extends proximally from the proximal end of the main body. The vaporizer 5 is structured so that when a user inhales through the mouthpiece 19, air is drawn into the device 5 through the air inlets 12 and into a vaporization chamber 14 via an internal flow path. At the same time, a heating coil 15 disposed within the vaporization chamber 14 is activated. The heating coil 15 heats the air in the chamber 14 along with vaporizable liquid drawn from the liquid reservoir 16 by a wicking material 17. The resulting combination of air and vapor is drawn through a chimney 18 to the mouthpiece 13 and out through an exit port 20.

In this and other personal vaporizers, there is a potential for residual liquid to be retained in the vaporization chamber when the heating coil is deactivated. There is also a potential for further liquid to migrate through the wick into the chamber due to changes in pressure, temperature or other atmospheric conditions or due to rough handling or improper use. In either case, when the vaporizer is tilted vertically or stood on one end, the liquid in the chamber will tend to flow through the air passages toward either the distal or proximal end of the device. Liquid passing into the air inlet section may then leak out through the air inlets. Liquid passing into the mouthpiece can leak out through the mouthpiece exit.

It will be understood that there are many other vaporizer configurations, but all have the general configuration of one or more air inlets upstream of a vaporization chamber and one or more exit ports downstream of the vaporization chamber. In some configurations, the air inlet port or ports may provide a direct flow path into the vaporization chamber. In other configurations, the air flow path from the air inlet ports to the vaporization chamber may comprise one or more intermediate passageways and/or chambers.

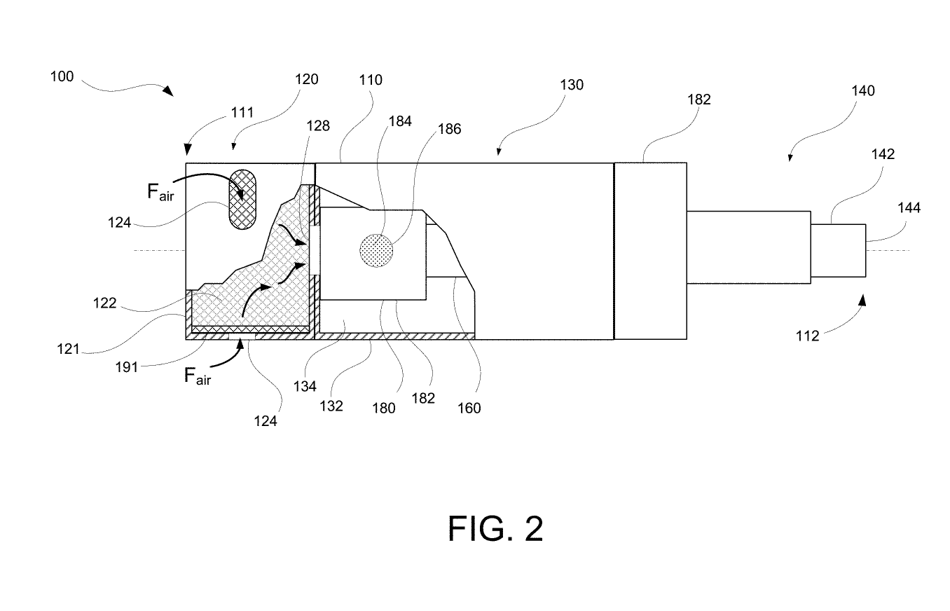

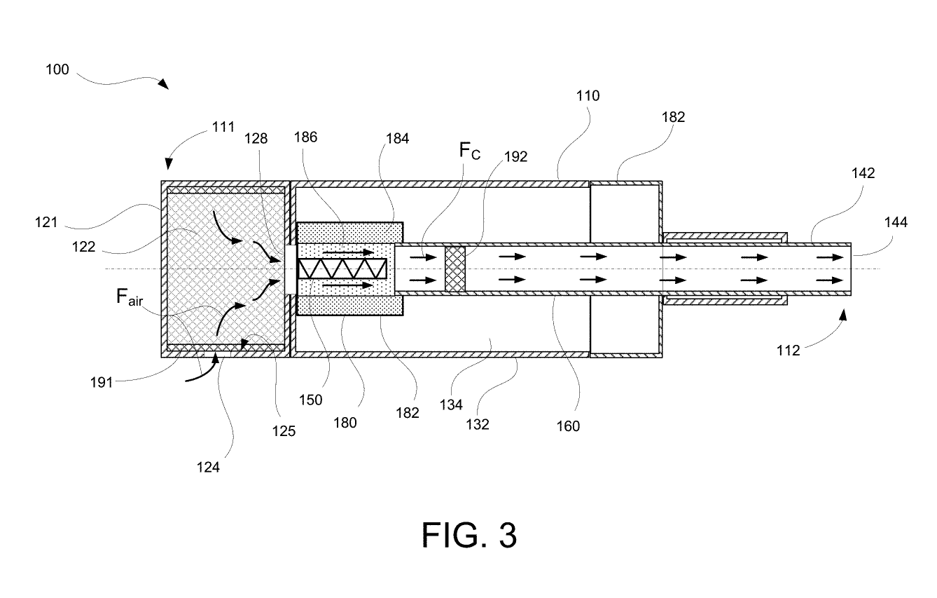

FIGS. 2 and 3 provide a schematic depiction of a personal vaporizer 100 according to an illustrative aspect of the invention. The personal vaporizer 100 has a configuration similar to the vaporizer shown in FIG. 1. The vaporizer 100 comprises a cylindrical main body 110 having an air inlet section 120 defining a distal device end 111, a reservoir/vaporization section 130, and a cap 182. A mouthpiece section 140 extends proximally from the cylindrical main body 110. The mouthpiece section 140 comprises a mouthpiece 142 defining a proximal device end 112 and an exit port 144.

The reservoir/vaporization section 130 includes a liquid reservoir 132 in which is disposed a vaporizable liquid 134. The liquid reservoir 130 may be configured as a simple tank in which the liquid 134 is disposed. In some embodiments, the reservoir 130 may comprise an adsorptive or absorptive material or structure that retains the vaporizable liquid 134. A liquid transport structure 180 is configured and positioned to be in contact with the liquid 134 in the reservoir 132 and for drawing the liquid 134 out of the reservoir 132. In the illustrated embodiment, the liquid transport structure 180 comprises a tubular wick structure 184 surrounded by a cylindrical case 182. An opening 186 in the case 182 allows fluid communication between the wick structure 184 and the liquid 134 in the reservoir 132. The tubular wick structure 184 defines a vaporization chamber 186 in which a heating element 150 is positioned. The wick structure 184 is configured to draw liquid 134 from the reservoir 132 into close proximity or in contact with the heating element 150. The heating element 150 may be configured to heat the vaporizable liquid through any conductive, convective, and/or radiative heat transfer mechanism. In typical vaporizers, the heating element 150 is or includes a resistance element in the form of a wire coil. In some cases, the resistance element is housed within a heat conductive casing. A chimney 160 extends between the vaporization chamber 186 and the mouthpiece 142 and defines a passageway for air and vaporization products to flow from the vaporization chamber 186 to the exit port 144.

The air inlet section 120 has a case wall 191 defining an inlet chamber 121. One or more air inlet ports 124 are formed through the case wall 191 to allow air to pass from the atmosphere into the inlet chamber 121. An inlet passageway 128 provides fluid communication between the inlet chamber 121 and the vaporization chamber 186. Flow through the vaporizer 100 is illustrated by arrows. Upstream of the vaporization chamber 186, the flow is essentially air (Fₐᵢᵣ). Downstream of the vaporization chamber 186, the flow is a combination of air and vaporization products (F_{C}).

While not shown in the drawings, the personal vaporizer 100 also includes a power source (e.g., a battery) in communication with the heating element 150 and a mechanism for selectively activating the heating element 150.

The personal vaporizer 100 also includes an upstream liquid barrier 191 configured and positioned to inhibit or prevent the vaporizable liquid 134 (or other target liquid) from flowing out through the air inlets 124 when the vaporizer is not in use. In this way, leakage of vaporizable liquid out through the collective inlet portal is substantially reduced or prevented. The liquid barrier 191 is formed from an air permeable medium so that air can still flow from the air inlets 124 to the vaporization chamber 186 when a pressure differential (draw force) is applied by a user inhaling at the exit port 144. The air permeable medium may be a sheet-like cloth, screen, or perforated membrane or may be a substantially three dimensional body having passageways (e.g., tortuous flow paths) formed there-through.

The air permeable medium may be selected so as to provide the desired liquid flow inhibition when the device is not in use while minimizing the effect on air flow during use. The medium preferably has passages sized so that the viscosity of the vaporizable liquid prevents the liquid from passing upstream from one side of the medium to the other when a typical flow potential is applied (e.g., due to gravity or jostling of the device). The vaporizable liquids used in personal vaporizers have a wide range of viscosities. Some have viscosities on the order of 1.0-1.8 mPa-sec at non-operating temperatures (e.g., 0-20° C.) and 0.01-0.4 mPa-sec at operating temperatures (e.g., 100-600° C.), which are little different from those of water. More viscous liquids, however, may have viscosities above 1000 mPa-sec at operating temperatures and above 10,000 mPa-sec at non-operating temperatures.

It can readily be seen that the passageways of the air permeable medium can be made larger for higher viscosity liquids. Lower viscosity liquids, however, require smaller pore or other passageway sizes. Making these passages too small, however, can result in a significant impedance to air flow during operation. Ideally, the air permeable medium allows air to pass through with little or no impedance when a typical pressure differential is applied (e.g., due to inhalation by a user at the exit port 144). Depending on the vaporizable liquid, the passageway size of the medium may be large enough that there is no significant increase in air flow impedance. In some cases, however, there may be a trade-off between liquid inhibition and air flow impedance. How the porous medium is tailored to handle this trade-off may depend on the type of personal vaporizer and/or the characteristics desired by the target user.

It is well-known that personal vaporizers can have widely varying flow and active material delivery characteristics. In some cases, such characteristics are the result of design. In others, they are simply the result of the scale or relative cost of manufacturing the device. In any case, the net result is that some personal vaporizers may deliver a high airflow rate and/or high active material delivery rate with a relatively moderate or low pressure differential (“draw”) applied by the user. Others may require a relatively high draw to attain the same airflow or delivery rate. Still others may be specifically configured to mimic the airflow and delivery characteristics of a cigarette.

In general, the airflow rate through any personal vaporizer is a function of the pressure differential applied by the user and the draw impedance (pressure drop) within the device. Devices having low draw impedance will deliver a relatively high flow rate for a small user-applied pressure differential. Devices having high draw impedance will produce a lower airflow rate for the same user-applied pressure differential.

The draw impedance of a personal vaporizer is generally a function of the ports, flow passages, and internal chambers of the device. The porous medium used in the liquid barrier can be tailored in combination with the geometry of the internal flow path to maintain or establish an overall draw impedance for the device while at the same time inhibiting the upstream flow of liquid. The change to the internal geometry of the device depends on the placement of the liquid barrier.

The porous medium used in liquid barriers of the invention may be formed from any suitable material having the desired air flow transmissibility, but with porosity or other limiting factors that inhibit the passage of liquid. The materials, porosity and thicknesses of the medium may be tailored to particular liquids. For example, for certain vaporizable liquids having relatively high viscosities, the medium may be or include a simple screen or mesh. The openings in such a screen may be sized so that the liquid's viscosity serves to inhibit its passage through the screen. Other structures that could be used include woven or non-woven cloth formed from polymer (man-made or natural) or metal fibers, perforated films or other membranes, and porous three dimensional structures, including but not limited to bonded or unbonded fiber structures and sintered plastic or metal structures. A particularly suitable material is a finely woven cloth formed from polyester monocomponent fibers. Examples of such a material include a range of products marketed by Saati S.p.A as Acoustex® and Saatifil Acoustex®, which are available with average pore sizes in a range from 18-285 μm.

The porous medium may also be formed from or comprise or be treated with a material that has properties geared toward repelling or attracting particular liquid materials. For example, material used for the porous medium may be formed from, include, or be treated with a hydrophobic or hydrophilic material. The use of a hydrophobic material, for example, would make it so that the liquid barrier would block the passage of a water-based liquid, but would assure that the liquid is not retained by the barrier, which would tend to reduce the area available for air-flow.

Turning back to the illustrated embodiment, the upstream liquid barrier 191 comprises a porous medium in the form of a sheet that is positioned around the entire inner circumferential surface 125 of the inlet chamber 121. As a result, the upstream liquid barrier 191 covers all of the air inlet ports 124 from the inside so that there is no opening from the inlet chamber 121 to the outside atmosphere that does not require passage through the barrier 191. Alternative embodiments may use a smaller, individual sheet of the barrier medium over each inlet port 124. Another alternative embodiment may include providing a single sheet of barrier material upstream of or within the passage 128 between the inlet chamber 121 and the vaporization chamber 186.

The sheet material used to form the barrier 191 can be any formable sheet having the desired pore size tailored to inhibit flow of the vaporizable liquid 134 while maintaining a desired air permeability (typically, but not exclusively, in a range of 1000 to 5000 L/m²-sec (at 20 mmWG)). The thickness of the sheet is preferably less than 500 μm. A desirable thickness of the sheet is in a range of 10 to 500 μm, with a particularly suitable thickness in a range of 10 to 200 μm. In a particular embodiment where the vaporizable liquid 134 has a viscosity profile similar to that of water, a suitable barrier sheet medium has an average pore diameter in a range of 20 to 30 μm and air permeability in a range of 2100 to 2800 L/m²-sec (at 20 mmWG). The barrier sheet material may be any of those previously discussed and may, in particular be a woven mesh formed from polyester monocomponent fibers. In a specific example of this embodiment, the barrier sheet material is Acoustex® 075, which has a thickness of 52 μm, an average pore diameter of 25 μm, and an air permeability of 2650 L/m²-sec (at 20 mmWG). Such a barrier sheet has been shown to be effective at preventing passage of a vaporizable liquid at typical operating temperatures for this type of device, but in particular at room temperature (15 to 25° C. It will be understood that the cumulative flow area of the air inlet ports (and, thus, the total flow area through the barrier sheet material) can be adjusted to optimize its impedance contribution or simply to provide a desired impedance contribution to the overall airflow impedance of the personal vaporizer.

The personal vaporizer 100 also includes a downstream liquid barrier 192 configured and positioned to inhibit liquid from passing (in either direction) between the exit passage 144 and the vaporization chamber 186 while allowing the passage of the combination of air and vaporization products drawn from the vaporization chamber to the exit passage 144 by a user. The downstream liquid barrier 191 may be configured to prevent passage of unvaporized vaporization liquid 134 which may otherwise pass to and through the exit passage 144 when the device is not in use. Toward that end, the characteristics of the downstream liquid barrier 192 could be similar to those of the upstream liquid barrier 191. In addition or instead, the downstream liquid barrier 192 may be configured to prevent external liquids (e.g., saliva or environmental moisture) from passing through the chimney 160 into the vaporization chamber.

The downstream barrier 192 may be formed from any suitable material having the desired air flow/vapor transmissibility, but with porosity or other limiting factors that inhibit passage of liquid. Like the upstream liquid barrier 191, the downstream barrier 192 may comprise materials tailored to particular liquids and/or may be optimized to provide a desired combination of liquid inhibition and air flow permeability. It may also be formed from or comprise a material that has properties geared toward repelling or attracting particular liquid materials (e.g., hydrophobic or hydrophilic materials).

In the illustrated embodiment, the downstream barrier 192 is formed as a disc positioned within the chimney 160. The exact positioning relative to the vaporization chamber 186 and the exit 144 may be selected based on the particular application. In a particular embodiment, the downstream barrier 192 may be or comprise one or more sheets of material similar to that described above for the upstream barrier 191.

In an alternative embodiment, more than one downstream liquid barrier may be used. In particular variations of such an embodiment, the downstream barriers may have different affinity characteristics. For example, a downstream-most barrier may be hydrophilic so as to retain external moisture and prevent it from passing back out through the exit portal, while a barrier closer to the vaporization chamber could be hydrophobic to prevent retention of vaporizable liquid.

It will be understood that the flow characteristics of the upstream and downstream liquid barriers 191, 192 may be collectively designed along with internal flow geometries to provide a desired overall air flow impedance for the vaporizer 100, which can be tailored to particular user experiences such as those described above.

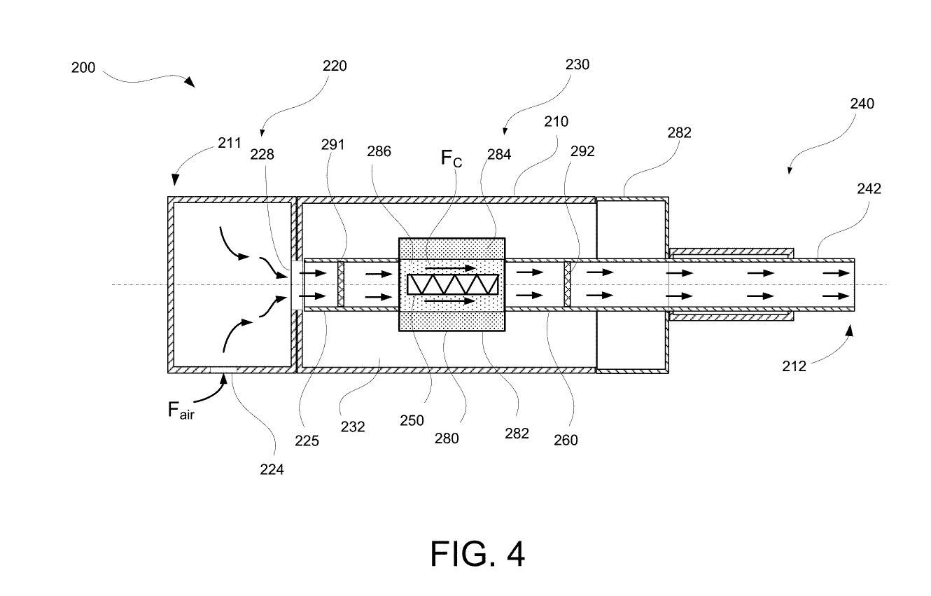

It will also be understood that the liquid barriers of the invention may be placed anywhere within the flow paths upstream or downstream of the vaporization chamber. FIG. 4 provides a schematic depiction of a personal vaporizer 200 according to another illustrative aspect of the invention in which an upstream liquid barrier is positioned within an airflow duct just upstream of the vaporization chamber. The personal vaporizer 200 has a configuration that is generally similar to the vaporizer of FIGS. 2 and 3. It comprises a cylindrical body 210 having an air inlet section 220 defining a distal end 211, a reservoir/vaporization section 230, and a cap 282. A mouthpiece section 240 extends proximally from the cylindrical body 210. The mouthpiece section 240 comprises a mouthpiece 242 defining a proximal end 212 and an exit port 244.

The reservoir/vaporization section 230 includes a liquid reservoir 232 in which is disposed a vaporizable liquid 234. The liquid reservoir 230 may be configured as a simple tank in which the liquid 234 is disposed. In some embodiments, the reservoir 230 may comprise an adsorptive or absorptive material or structure that retains the vaporizable liquid 234. A liquid transport structure 280 is configured and positioned to be in contact with the liquid 234 in the reservoir 232 and for drawing the liquid 234 out of the reservoir 232. In the illustrated embodiment, the liquid transport structure 280 comprises a tubular wick structure 284 surrounded by a cylindrical case 282. The tubular wick structure 284 defines a vaporization chamber 286 in which a heating element 250 is positioned. The wick structure 284 is configured to draw liquid 234 from the reservoir 232 into close proximity or in contact with the heating element 250. The heating element 250 may be configured to heat the vaporizable liquid through any conductive, convective, and/or radiative heat transfer mechanism. In typical vaporizers, the heating element 250 is or includes a resistance element in the form of a wire coil. In some cases, the resistance element is housed within a heat conductive casing. A chimney 260 extends between the vaporization chamber 286 and the mouthpiece 242 and defines a passageway for air and vaporization products to flow from the vaporization chamber 286 to the exit port 244.

The air inlet section 220 has a case wall 291 defining an inlet chamber 221. One or more air inlet ports 224 are formed through the case wall 291 to allow air to pass from the atmosphere into the inlet chamber 221. An inlet passageway 228 provides fluid communication between the inlet chamber 221 and an air conduit 225 that flows into the vaporization chamber 286. Flow through the vaporizer 200 is illustrated by arrows. Upstream of the vaporization chamber 286, the flow is essentially air (Fₐᵢᵣ). Downstream of the vaporization chamber 286, the flow is essentially a combination of air and vaporization products (F_{C}).

While not shown in the drawings, the personal vaporizer 200 also includes a power source (e.g., a battery) in communication with the heating element 250 and a mechanism for selectively activating the heating coil

The personal vaporizer 200 also includes an upstream liquid barrier 291 positioned in the air conduit 225 and a downstream liquid barrier 292 positioned in the chimney 260. The liquid flow and other characteristics of the upstream and downstream liquid barriers 291, 292 may be substantially similar to those previously described.

It will be understood that personal vaporizers according to the invention may have either or both of the upstream and downstream liquid barriers. It will also be understood that if both upstream and downstream barriers are used, the two barriers may be formed from the same or different materials, may have the same or different flow characteristics, and may have the same or different liquid affinity characteristics. As before, the media used in either or both of the liquid barriers 291, 292 may be selected to provide a desired combination of liquid inhibition and airflow permeability or impedance. In particular, the flow characteristics of the liquid barriers 191, 192 may be collectively designed along with the internal geometries of the air conduit 225 and the chimney 260 to provide a desired overall air flow impedance (i.e., draw resistance) for the vaporizer 100.

The leak prevention methods and materials of the invention may be used in virtually any personal vaporizer, including those described in U.S. application Ser. No. 15/639,139, filed Jun. 30, 2017 and U.S. Prov. App. No. 62/580,490, filed Nov. 2, 2017, the complete disclosures of which are incorporated herein by reference in their entirety. In addition, personal vaporizers incorporating the upstream and/or downstream liquid barriers of the invention may be configured to provide or maintain any set of desired flow and delivery characteristics regardless of scale or desired airflow versus draw regime.

While the foregoing illustrates and describes exemplary embodiments of this invention, it is to be understood that the invention is not limited to the construction disclosed herein. The invention can be embodied in other specific forms without departing from the spirit or essential attributes.

What is claimed is:

1. A micro-vaporizer comprising:
a main body having a main body interior;
a vaporization chamber disposed within the main body interior;
a vaporizable liquid reservoir disposed within the main body configured for selectively retaining a vaporizable liquid;
a heating element disposed within the vaporization chamber and configured to selectively heat and vaporize vaporizable liquid drawn from the liquid reservoir;
one or more air inlet openings in the main body collectively defining an air intake portal;
an air flow passage from the air intake portal to the vaporization chamber, the air flow passage providing a first fluid communication path between the vaporization chamber and an ambient environment external to the main body;
a vaporization products flow passage from the vaporization chamber to an exit port, the vaporization products flow passage providing a second fluid communication path between the vaporization chamber and the ambient environment external to the main body; and
a first air-permeable liquid barrier disposed within the air flow passage, the first air-permeable liquid barrier being configured to inhibit passage of the vaporizable liquid through the air flow passage.

2. A micro-vaporizer according to claim 1 wherein the first air-permeable liquid barrier comprises a porous medium having a plurality of flow passages formed therethrough.

3. A micro-vaporizer according to claim 2 wherein the porous medium is one of the set consisting of a perforated membrane, a bonded mesh, a three dimensional fiber structure, a sintered metal structure, and a sintered plastic structure.

4. A micro-vaporizer according to claim 2 wherein the porous medium is a woven or non-woven cloth.

5. A micro-vaporizer according to claim 4 wherein the woven or non-woven cloth is formed from polymer fibers.

6. A micro-vaporizer according to claim 5 wherein the polymer fibers comprise polyester fibers.

7. A micro-vaporizer according to claim 4 wherein the woven or non-woven cloth has an average pore size in a range of 10 μm to 100 μm.

8. A micro-vaporizer according to claim 4 wherein the woven or non-woven cloth has an average pore size in a range of 20 μm to 30 μm.

9. A micro-vaporizer according to claim 2 wherein the porous medium is selected and configured to provide a predetermined air permeability level.

10. A micro-vaporizer according to claim 2 wherein the porous medium has an air permeability level in a range of 1000 to 5000 L/m²-sec at 20 mmWG.

11. A micro-vaporizer according to claim 2 wherein the porous medium has an air permeability level in a range of 2100 to 2800 L/m²-sec at 20 mmWG.

12. A micro-vaporizer according to claim 2 wherein the porous medium has an air permeability level of 2650 L/m²-sec at 20 mmWG.

13. A micro-vaporizer according to claim 2 wherein the porous medium maintains its structural integrity at an operating temperatures up to 600° C.

14. A micro-vaporizer according to claim 2 wherein the porous medium comprises at least one of the set consisting of a hydrophobic material and a hydrophilic material.

15. A micro-vaporizer according to claim 1 wherein the first liquid barrier is effective to inhibit passage of a vaporizable liquid having a viscosity in a range of 1.0 mPa-sec to 1.8 mPa-sec at temperatures in a range of 0° C. to 20° C.

16. A micro-vaporizer according to claim 1 further comprising
a second air-permeable liquid barrier disposed within the vaporization products flow passage, the second air-permeable liquid barrier being configured to inhibit passage of the vaporizable liquid through the vaporization products flow passage.

17. A micro-vaporizer according to claim 16 wherein the second air-permeable liquid barrier comprises a second porous medium having different flow properties from the first porous medium.

18. A micro-vaporizer according to claim 17 wherein the second porous medium comprises one of the set consisting of a hydrophobic material and a hydrophilic material.

19. A micro-vaporizer according to claim 16 wherein the second air-permeable liquid barrier comprises a plurality of spaced apart porous media.

20. A micro-vaporizer according to claim 1
wherein the main body comprises an air inlet section having an inlet section wall through which the one or more air inlet openings are formed, the inlet section wall having an inner wall surface defining an air inlet chamber that is part of the air flow passage, and
wherein the first liquid barrier comprises a sheet of a porous medium disposed within the inlet chamber against the inner wall surface so as to cover at least one of the one or more air inlet openings.

21. A micro-vaporizer according to claim 17 wherein the inner wall surface is cylindrical and the porous medium sheet is sized and positioned to cover a full circumferential section of the inner wall surface including all of the one or more air inlet openings.

22. A micro-vaporizer according to claim 17 wherein the first liquid barrier comprises a plurality of sheets of a porous medium each disposed within the inlet chamber against the inner wall surface so as to cover at least one of the one or more air inlet openings, the plurality of sheets collectively covering all of the one or more air inlet openings.

23. A micro-vaporizer according to claim 17 wherein the porous medium is a woven or non-woven cloth.
